(19) United States
(12) Patent Application Publication
(10) Pub. No.: US 2026/0171225 A1
(43) Pub. Date: Jun. 18, 2026

(54) Systems and Methods of Identifying Healthcare Conditions Across Multiple Separate Healthcare Computer Networks

(71) Applicant: Dicerra Safety Systems Inc., Ottawa (CA)

(72) Inventors: Theon Te Koeti, Ottawa (CA); Thomas Allen, Halifax (CA)

(73) Assignee: Dicerra Safety Systems Inc., Ottawa (CA)

(21) Appl. No.: 18/982,524

(22) Filed: Dec. 16, 2024

(51) Int. Cl. G16H 40/20; G06N 20/00

(52) U.S. Cl. CPC G16H 40/20; G06N 20/00

(57) ABSTRACT

Systems and methods of identifying healthcare conditions across multiple separate healthcare computer networks are provided. The system can obtain first clinical data from at least one computing device of a first computer network and second clinical data from at least one computing device of a second computer network, the second computer network separate from the first computer network. The system can train, using the first clinical data and the second clinical data, a machine learning model to identify a plurality of healthcare conditions. The system can receive a dataset indicative of a healthcare service. The system can determine, using the machine learning model, that the dataset indicative of the healthcare service correlates to a first healthcare condition of the plurality of healthcare conditions. The system can cause a first computing device of the first computer network and a second computing device of the second computer network to perform an action.

### BACKGROUND

Data structures can be input to, stored in, and retrieved from data processing networks. The data structures can be contained with a network or unavailable to other data networks.

### SUMMARY

At least one aspect is directed to a system of identifying healthcare conditions common to multiple separate healthcare computer networks. The system can include one or more processors coupled with memory. The system can obtain from at least one computing device of a first computer network, first clinical data. The system can obtain, from at least one computing device of a second computer network, second clinical data. The second computer network can be separate from the first computer network. The system can train, using the first clinical data and the second clinical data, a machine learning model to identify a plurality of healthcare conditions. The system can receive, from the first computer network or the second computer network, a dataset indicative of a healthcare service. The system can determine, using the machine learning model, that the dataset indicative of the healthcare service correlates to a first healthcare condition of the plurality of healthcare conditions. The system can cause, responsive to determining that the dataset indicative of the healthcare service correlates to the first healthcare condition, a first computing device of the first computer network and a second computing device of the second computer network to perform an action.

At least one aspect is directed to a method of identifying healthcare conditions common to multiple separate healthcare computer networks. The method can include obtaining, by one or more processors from at least one computing device of a first computer network, first clinical data. The method can include obtaining, by one or more processors from at least one computing device of a second computer network, second clinical data. The second computer network can be separate from the first computer network. The method can include training, by the one or more processors using the first clinical data and the second clinical data, a machine learning model to identify a plurality of healthcare conditions. The method can include receiving, by the one or more processors from the first computer network or the second computer network, a dataset indicative of a healthcare service. The method can include determining, by the one or more processors using the machine learning model, that the dataset indicative of the healthcare service correlates to a first healthcare condition of the plurality of healthcare conditions. The method can include causing, by the one or more processors responsive to determining that the dataset indicative of the healthcare service correlates to the first healthcare condition, a first computing device of the first computer network and a second computing device of the second computer network to perform an action.

### BRIEF DESCRIPTION OF THE DRAWINGS

The accompanying drawings are not intended to be drawn to scale. Like reference numbers and designations in the various drawings indicate like elements. For purposes of clarity, not every component can be labeled in every drawing. In the drawings:

FIG. 1 depicts an example system of identifying healthcare conditions common to multiple separate healthcare computer networks.

FIG. 2 depicts an example system of identifying healthcare conditions common to multiple separate healthcare computer networks.

FIG. 3 depicts an example system of identifying healthcare conditions common to multiple separate healthcare computer networks.

FIG. 4 depicts an example method of identifying healthcare conditions common to multiple separate healthcare computer networks.

FIG. 5 depicts an example system of identifying healthcare conditions common to multiple separate healthcare computer networks.

### DETAILED DESCRIPTION

Following below are more detailed descriptions of various concepts related to, and implementations of multi-network communication to identify conditions such as common healthcare conditions as indicated by siloed data in individual, separate computer networks. The various concepts introduced above and discussed in greater detail below can be implemented in any of numerous ways.

Systems and methods described herein relate to a data processing system that can train or execute a machine learning model to identify healthcare conditions based on data or data structures obtained from multiple separate healthcare computer networks that may otherwise not communicate with each other. The systems and methods described herein can obtain information relating to healthcare conditions from multiple separate healthcare computer networks and can identify trends in healthcare conditions that could otherwise be undetected within a single computer network. The systems and methods described herein can determine from trends and patterns identified in the multi-network data what is working best in each healthcare computer network, and can transmit data structures to cause computers across multiple separate healthcare computer networks to perform various actions. The actions performed by the computing devices of the separate healthcare computer networks can indicate what healthcare treatment options are or are not working in each healthcare computer network, and can recommend that certain actions be taken, avoided, changed, or deferred.

Separate healthcare computer networks may not share information with each other due to the desire to maintain patient and clinician privacy. As a result, a healthcare condition that can happen infrequently at one healthcare computer network but frequently when observing multiple separate healthcare computer networks can go unnoticed or unresolved, resulting in inefficiencies within the multiple separate healthcare computer networks.

To resolve these and other challenges, systems and methods to identify healthcare conditions across multiple separate healthcare computer networks are provided as described herein. The system can include one or more processors coupled with memory. The system can obtain from at least one computing device of a first computer network, first clinical data. The system can obtain, from at least one computing device of a second computer network, second clinical data. The second computer network can be separate from the first computer network. The system can train, using the first clinical data and the second clinical data, a machine learning model to identify a plurality of healthcare conditions. The system can receive, from the first computer network or the second computer network, a dataset indicative of a healthcare service. The system can determine, using the machine learning model, that the dataset indicative of the healthcare service correlates to a first healthcare condition of the plurality of healthcare conditions. The system can cause, responsive to determining that the dataset indicative of the healthcare service correlates to the first healthcare condition, a first computing device of the first computer network and a second computing device of the second computer network to perform an action.

Each separate healthcare computer network may use an individual system to separately identify healthcare conditions only within said healthcare computer network. Systems and methods described herein improve on these individual systems by providing a singular machine learning model to determine that information from the separate healthcare computer networks correlate to a healthcare condition. As a result, less computations may need to be performed to identify the healthcare conditions, which can reduce the amount of computational resources including processing power, electrical power, memory usage, or computation time compared to each healthcare computer network using an individual system. Additionally, the machine learning model can determine the healthcare conditions based on a larger dataset including information from each separate healthcare computer network, allowing for more accurate identification of the healthcare conditions.

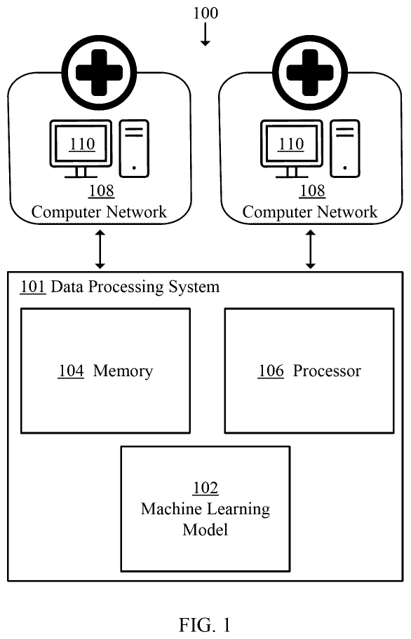

FIG. 1, among others, depicts an example of a system 100 of identifying healthcare conditions common to multiple separate healthcare computer networks (e.g., computer network 108). System 100 can include at least one data processing system 101. Data processing system 101 can communicate with at least one computing device 110 of at least one computer network 108. For example, data processing system 101 can receive data from a first computing device 110 of a first computer network 108. Data processing system can include at least one memory 104, at least one processor 106, or at least one machine learning model 102.

System 100 can include at least one machine learning model 102. Machine learning model 102 can include, for example and without limitation, one or more language models, LLMs, attention-based neural networks, transformer-based neural networks, generative pretrained transformer (GPT) models, bidirectional encoder representations from transformers (BERT) models, encoder/decoder models, sequence to sequence models, autoencoder models, generative adversarial networks (GANs), convolutional neural networks (CNNs), recurrent neural networks (RNNs), diffusion models (e.g., denoising diffusion probabilistic models (DDPMs)), among others or various combinations thereof.

For example, machine learning model 102 can include at least one GPT model. The GPT model can receive an input sequence and can parse the input sequence to determine a sequence of tokens (e.g., words or other semantic units of the input sequence, such as by using Byte Pair Encoding tokenization). The GPT model can include or be coupled with a vocabulary of tokens, which can be represented as a one-hot encoding vector, where each token of the vocabulary has a corresponding index in the encoding vector; as such, the GPT model can convert the input sequence into a modified input sequence, such as by applying an embedding matrix to the token tokens of the input sequence (e.g., using a neural network embedding function), or applying positional encoding (e.g., sin-cosine positional encoding) to the tokens of the input sequence. The GPT model can process the modified input sequence to determine a next token in the sequence (e.g., to append to the end of the sequence), such as by determining probability scores indicating the likelihood of one or more candidate tokens being the next token, and selecting the next token according to the probability scores (e.g., selecting the candidate token having the highest probability scores as the next token). For example, the GPT model can apply various attention or transformer based operations or networks to the modified input sequence to identify relationships between tokens for detecting the next token to form the output sequence.

System 100 can include at least one memory 104. Memory 104 can include one or more devices (e.g., memory units, memory devices, storage devices, etc.) for storing data or computer code for completing or facilitating the various processes described in the present disclosure. Memory 104 can include random access memory (RAM), read-only memory (ROM), hard drive storage, temporary storage, non-volatile memory, flash memory, optical memory, or any other suitable memory for storing software objects or computer instructions. Memory 104 can include database components, object code components, script components, or any other type of information structure for supporting the various activities and information structures described in the present disclosure. Memory 104 can be communicably connected to at least one processor 106 and can include computer code for executing (e.g., by processor 106) one or more processes described herein. For example, memory 104 can include computer code for executing machine learning model 102 by processor 106.

System 100 can include at least one processor 106. Processor 106 can implement various components of system 100 or portions thereof. Processor 106 can be coupled with at least one memory 104. Processor 106 can include a general purpose or specific purpose processors, an application specific integrated circuit (ASIC), one or more field programmable gate arrays (FPGAs), a group of processing components, or other suitable components. Processor 106 can execute computer code or instructions stored in memory 104 or received from other computer readable media (e.g., CDROM, network storage, a remote server, etc.). Processor 106 can be configured in various computer architectures, such as graphics processing units (GPUs), distributed computing architectures, cloud server architectures, client-server architectures, or various combinations thereof. One or more first processors 106 can be implemented by a first device, such as an edge device, and one or more second processors 106 can be implemented by a second device, such as a server or other device that is communicatively coupled with the first device and can have greater processor or memory resources.

System 100 can include at least one computer network 108. Computer network 108 can include the Internet, local, wide, metro, or other area networks, intranets, satellite networks, and other communication networks such as voice or data mobile telephone networks. Computer network 108 can be used to access information resources such as web pages, web sites, streaming media resources, domain names, or uniform resource locators that can be provided, output, rendered, or displayed on at least one computing device 110.

Computer network 108 can be or include one or more healthcare computer networks. The healthcare computer networks can include at least one hospital or healthcare facility. Each healthcare computer network 108 can include one or more computing devices 110 networked to communicate with each other in a healthcare setting. For example, computing devices 110 that form at least part of computer network 108 can include and transmit data subject to privacy or confidentiality protections so that the other computing devices 110 that are part of the same computer network 108 can access this data, but computing devices 110 not connected to this computer network 108 cannot access this data. The computing devices 110 can be prohibited from providing personally identifiable or other data outside of the computer network 108, such as to a second, separate computer network 108. Each hospital or healthcare facility can include a variety of healthcare services such as primary care or specialized medical services. For example, a healthcare computer network can include a general hospital and a cardiac specialist hospital. Each hospital or healthcare facility can employ a number of clinicians. These clinicians can perform a variety of duties at a specific hospital or healthcare facility or across a variety of hospitals or healthcare facilities within the network. Each healthcare computer network can include data, which can be gathered during the day-to-day operations of the hospitals or healthcare facilities or entered by the clinicians or other employees. The data can be stored in computer network 108.

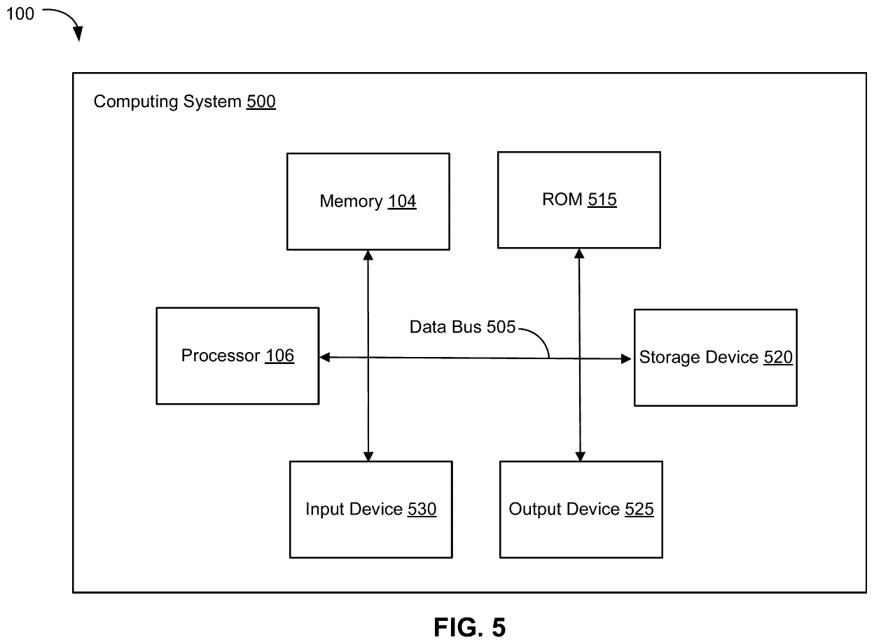

System 100 can include at least one computing device 110. Computing device 110 can include a desktop computer, laptop computer, tablet computer, smart phone, mobile telecommunication device, or portable computer. Computing device 110 can include one or more components depicted in FIG. 5. Computing device 110 can communicate with computer network 108 through wired or wireless communications. Computer network 108 can include at least one computing device 110. For example, a healthcare computer network can include hundreds of computing devices 110 across all associated hospitals or healthcare facilities. Computing device 110 can be accessed by various employees of the associated healthcare computer network, such as clinicians, to enter data.

Computer network 108 can include a first computer network 108 and a second computer network 108. First computer network 108 can be included in a first healthcare computer network and second computer network 108 can be included in a second healthcare computer network. The first healthcare computer network can be separate from the second healthcare computer network. For example, the first healthcare computer network can include a first set of hospitals and the second healthcare computer network can include a second set of hospitals. The first set of hospitals not including any hospital of the second set of hospitals and the second set of hospitals not including any hospital of the first set of hospitals. Data stored in first computer network 108 can be inaccessible to second computer network 108 and data stored in second computer network 108 can be inaccessible to first computer network 108.

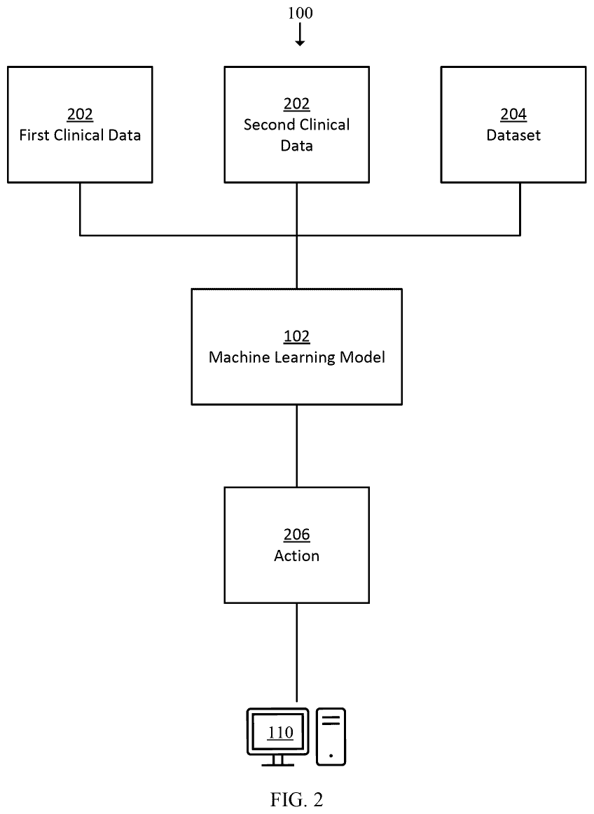

FIG. 2, among others, depicts an example of a system 100 of identifying healthcare conditions common to multiple separate healthcare computer networks (e.g., computer network 108). System 100 can obtain at least one clinical data 202 from at least one computing device 110 of at least one computer network 108. For example, first clinical data 202 can be obtained from at least one computing device 110 of first computer network 108 and second clinical data 202 can be obtained from at least one computing device 110 of second computer network 108.

Clinical data 202 can include data stored in computer network 108 or stored in another database accessible to system 100. Clinical data 202 can be recorded in computer network 108 by a clinician or employee of a healthcare computer network associated with computer network 108. Clinical data 202 can include patient data, administrative data, clinician's notes, hospital records, and other various data. For example, clinical data 202 can include medical history of patients, including information about past illnesses, surgeries, allergies, family medical history, lab results, current treatment plans, or imaging results (e.g., x-ray, MRI imaging, CT scans, ultrasounds, etc.). Data processing system 101 anonymize the clinical data 202, for example to remove personal identifiable information regarding any patient or clinician associated with the data, and to comply with any data privacy requirements. Clinical data 202 can include clinician's notes on prescribed drugs, dosages, administration schedules, detailed accounts of surgeries performed, ongoing observations, patient updates, instructions, diagnoses, recommendations, recorded vital signs of patients, or lab results. Clinical data 202 can also include consent forms signed by patients, appointment information, billing information, or insurance information.

Clinical data 202 can include a data format. The format can include textual data, numeric data, image data, voice data, and multimedia data. For example, clinical data 202 can include a clinician's voice documentation on a patient's condition. Clinical data 202 can include a written description of a patient's prescribed medications.

Clinical data 202 can include first clinical data 202 and second clinical data 202. First clinical data 202 can include data stored in first computer network 108 and second clinical data 202 can be stored in second computer network 108. First clinical data 202 can be inaccessible to first computer network 108 and second clinical data 202 can be inaccessible to first computer network 108. First clinical data 202 can have a first data format and second clinical data 202 can have a second data format different from the first data format. For example, first clinical data 202 can include voice data and second clinical data 202 can include textual data.

System 100 can convert at least one clinical data 202 from a first data format to a second data format. For example, clinical data 202 can be converted from voice data to textual data. The second data format can include select information from first data format. Key information from clinical data 202 in the first data format can be identified, extracted, and used to populate the second data format. Second data format can include less information than the first data format. Second data format can exclude sensitive information such as a patient's or a hospital's name. For example, clinical data 202 in the first data format can state, “A dosage of a medication was given to Mr. John Doe on December 23^{rd} at 9:34 AM. Mr. John Doe had an adverse reaction to the medication on December 23^{rd} at 10:01 AM.” Clinical data 202 in the second data format can state, “Patient received a dosage of a medication on December 23^{rd} at 9:34 AM. Patient had an adverse reaction to the dosage of the medication on December 23^{rd} at 10:01 AM.”

System 100 can train at least one machine learning model 102 to identify a plurality of healthcare conditions. The plurality of healthcare conditions can include various data trends across the healthcare computer networks associated with computer networks 108. For example, healthcare conditions can include patient admissions, discharges, diagnosis trends, treatment effectiveness, mortality rates, patient satisfaction. Healthcare conditions can include bed occupancy rates, wait times, staffing levels, on-duty staff, and number of different treatments being performed. Healthcare conditions can include hospital revenue, cost analysis, insurance claim denials, and infection rates. Healthcare conditions can include the results of various events, such as effectiveness of medications, treatments, surgeries. Healthcare conditions can include trends of various incidents such as, medication errors, surgical complications, incompliance with protocols, in hospital accidents (e.g., patient falling out of bed), and unexpected reactions to treatments. For example, the plurality of healthcare conditions can include a data trend across the healthcare computer network associated with computer networks 108 of clinicians accidentally administering medication A when they mean to administer medication B.

Clinical data 202 can be used to train machine learning model 102. For example, machine learning model 102 can be configured using various unsupervised or supervised training operations. Machine learning model 102 can be configured using training data from various domain-agnostic or domain-specific data sources, including but not limited to various forms of text, speech, audio, image, or video data, or various combinations thereof (e.g., clinical data 202). The training data can include a plurality of training data elements (e.g., training data instances). Each training data element can be arranged in structured or unstructured formats; for example, the training data element can include an example output mapped to an example input, such as a query representing a service request or one or more portions of a service request, and a response representing data provided responsive to the query. The training data can include data that is not separated into input and output subsets (e.g., for configuring machine learning model 102 to perform clustering, classification, or other unsupervised ML operations). The training data can include human-labeled information, including but not limited to feedback regarding outputs the system. This can allow the system to generate more human-like outputs.

For example, machine learning model 102 can be trained with first clinical data 202 or second clinical data 202. One or more loss functions (e.g., mean absolute error, mean squared error, root mean squared error, etc.) can be utilized during training to set the weights or biases of machine learning model 102. Multiple iterations of training can be performed for machine learning model 102 until a predetermined accuracy or precision is achieved by the models to ensure accurate performance of machine learning model 102.

System 100 can receive at least one dataset 204. Dataset 204 can be received from at least one computer network 108. Dataset 204 can be recorded by a clinician or employee within the healthcare computer network associated with computer network 108. Dataset 204 can be indicative of a healthcare service. The healthcare service can include at least one event or a series of events. For example, the healthcare service can include clinician's notes on surgeries, treatment plans, imaging results, test results, and distributing medications. The healthcare service can also include various incidents such as, medication errors, surgical complications, incompliance with protocols, hospital accidents (e.g., a patient falling out of bed), and unexpected reactions to treatments.

Dataset 204 can include textual data, numeric data, image data, voice data, and multimedia data. For example, dataset 204 can include a clinician's voice documentation on a patient's condition. Dataset 204 can include a written description of a patient's prescribed medications.

Dataset 204 can include at least part of or none of clinical data 202. Clinical data 202 can include data spanning a first time period and dataset 204 can include data spanning a second time period. The first time period can occur temporally prior to the second time period. For example, clinical data 202 can include historic information from a healthcare computer network from the time the healthcare computer network began to the time machine learning model 102 is trained. Dataset 204 can then include data occurring after the training of machine learning model 102.

A first dataset 204 can be received from first computer network 108 and a second dataset 204 can be received from second computer network 108. First dataset 204 and second dataset 204 can be indicative of the same healthcare service. First dataset 204 can be indicative of a first healthcare service. Second dataset 204 can be indicative of a second healthcare service. The first healthcare service can be the same as (e.g., identical to) or different from the second healthcare service. For example, first healthcare service and second healthcare service can include an incident of a clinician distributing the wrong medication to a patient. First healthcare service can include a patient falling out of a bed and second healthcare service can include a patient having an allergic reaction to a medication.

Dataset 204 can be received from a different computer network 108 than clinical data 202. For example, first clinical data 202 can be received from a first computer network 108, second clinical data 202 can be received from a second computer network 108, and dataset 204 can be received from a third computer network 108, computer network 108 separate from first computer network 108 or second computer network 108.

System 100 can identify at least one data template based on dataset 204. The data template can be identified responsive to receiving dataset 204. For example, the data template can be associated with the healthcare service indicated by dataset 204. Data template can be selected from a set of data templates. Each data template of set of data template can be associated with different healthcare services. For example, a surgical complication healthcare service can have a first data template and a medication error healthcare service can have a second data template. Dataset 204 can be analyzed to determine at least one data template associated with the same healthcare service as indicated by dataset 204.

System 100 can extract key terms from dataset 204. The key terms extracted from dataset 204 can be determined based on the template selected for dataset 204. Each template can include various key terms. For example, a template for an allergic reaction can include key terms such as medication name, dosage, reaction, time medication was distributed, time reaction was recorded while a template for a surgical complication can include surgery type, description of complication, and cause of complication.

System 100 can populate at least one template with key terms from dataset 204. For example, key terms can be extracted from dataset 204 and then used to populate the template.

System 100 can identify information missing from dataset 204. For example, responsive to receiving dataset 204, information missing from dataset 204 can be identified. When extracting key terms from dataset 204 it can be determined that one or more key terms associated with the template are missing from dataset 204. For example, the template can include a time associated with the healthcare service, but dataset 204 can be missing a time associated with the healthcare service.

System 100 can transmit a request for the information missing from dataset 204 to computer network 108. For example, system 100 can transmit the request to first computer network 108 or second computer network 108. The request can be sent to the same computer network 108 dataset 204 was received from. For example, dataset 204 can be received from first computer network 108 and the request can be sent to first computer network 108. The request can be sent to a different computer network 108 than the one dataset 204 was received from. For example, dataset 204 can be received from first computer network 108 and the request can be sent to second computer network 108.

The request for the missing information can be in the form of a text request or an audio request. The request can specifically ask at least one clinician for the missing information. For example, the request can include a text request stating, “Please provide the date associated with the healthcare service.” The request can be associated with the template. For example, if a template for medication errors is identified the request can state, “What was the dosage of the medication provided,” which may not be a request if a template for surgical complications is identified.

System 100 can receive the information missing from dataset 204. The information missing from dataset 204 can be received responsive to requesting at least one computer network 108 for the information missing from dataset 204. The information missing from dataset 204 can be received from a different clinician than the clinician that entered the information in dataset 204. For example, a doctor can originally submit dataset 204 and a nurse can submit the information missing from dataset 204.

System 100 can populate dataset 204 with the information missing from dataset 204. For example, in response to receiving the information missing from dataset 204 from computer network 108, the information missing from dataset 204 can be populated into dataset 204. The template associated with dataset 204 can be populated with the information missing from dataset 204.

System 100 can determine, using machine learning model 102, that dataset 204 correlates to a first healthcare condition of the plurality of healthcare conditions. System 100 can determine, using machine learning model 102, that first dataset 204 and second dataset 204 correlate to a first healthcare condition of the plurality of healthcare conditions. First dataset 204 can correlate to a first healthcare condition of the plurality of healthcare conditions and second dataset 204 can correlate to a second healthcare condition of the plurality of healthcare conditions, the second healthcare condition different from or the same as (e.g., identical to) the first healthcare condition.

For example, system 100 can determine, using machine learning model 102, that first dataset 204 indicative of a first healthcare service and second dataset 204 indicative of the first healthcare service correlate to a first healthcare condition of the plurality of healthcare conditions. First dataset 204 indicative of a first healthcare service can correlate to a first healthcare condition of the plurality of healthcare conditions and second dataset 204 indicative of a first healthcare service can correlate to a second healthcare condition of the plurality of healthcare conditions. First dataset 204 indicative of a first healthcare service and second dataset 204 indicative of a second healthcare service can correlate to a first healthcare condition of the plurality of healthcare conditions. First dataset 204 indicative of a first healthcare service can correlate to a first healthcare condition of the plurality of healthcare conditions and second dataset 204 indicative of a second healthcare service can correlate to a second healthcare condition of the plurality of healthcare conditions.

System 100 can determine, using machine learning model 102, that the data template associated with at least one dataset 204 correlates to a first healthcare condition of the plurality of healthcare conditions.

System 100 can cause at least one computing device 110 of at least one computer network 108 to perform at least one action 206. For example, system 100 can cause first computing device 110 of first computer network 108 or second computing device 110 of a second computer network 108 to perform action 206. First computing device 110 of first computer network 108 can perform a first action 206 and second computing device 110 of second computer network 108 can perform a second action 206, first action 206 different than or the same as (e.g., identical to) second action 206. Action 206 can be caused responsive to determining that dataset 204 correlates to at least one healthcare condition. System 100 can cause a third computing device 110 of a third computer network 108 to perform action 206, third computer network 108 separate from first computer network 108 or second computer network 108. Third computer network 108 can be separate from computer network 108 clinical data 202 and dataset 204 are obtained and received from. For example, first clinical data 202 can be obtained from first computer network 108, dataset 204 can be received from second computer network 108, and system 100 can cause action 206 to be performed by third computer network 108. Action 206 can include one or more outputs (e.g., reports, documents, tables, charts, graphs, scripts, or images) provided for display via a graphical user interface of computing device 110.

Action 206 can be caused responsive to determining that the data template correlates to at least one healthcare condition. For example, in response to determining that the data template or dataset 204 correlates to the first healthcare condition, system 100 can cause first computing device 110 of first computer network 108 and second computing device 110 of second computer network 108 to perform action 206.

Action 206 can include a recommendation. Machine learning model 102 can determine that dataset 204 correlates to a first healthcare condition of the plurality of healthcare conditions. This can be determined by machine learning model 102 comparing dataset 204 to clinical data 202 and previously received datasets 204. Machine learning model 102 can generate the recommendation based on this comparison. The recommendation can be for at least one healthcare computer network associated with at least one computer network 108. For example, the recommendation can be for only a first healthcare computer network associated with a first computer network 108. The recommendation can be for a first healthcare computer network associated with a first computer network 108 and a second healthcare computer network associated with a second computer network 108. The recommendation can be for all healthcare computer networks associated with computer networks 108. The recommendation can be provided for display on at least one computer network 108. For example, the recommendation can be provided for display on first computer network 108. The recommendation can be provided for display from one of first computer network 108 or second computer network 108.

The recommendation can be displayed to minimize an occurrence of the healthcare service or the healthcare condition. For example, dataset 204 can be indicative of a healthcare service of a patient having an unexpected reaction to medication X. Machine learning model 102 can determine that dataset 204 correlates to a first healthcare condition of multiple patients having an unexpected reaction to medication X. After identifying the trend, machine learning model 102 can analyze dataset 204, clinical data 202, and previously received datasets 204 to determine the cause of the first healthcare condition. For example, machine learning model 102 can determine that each patient who had an unexpected reaction to medication X was also taking medication Y. Machine learning model 102 can determine that taking both medication Y and medication X can cause the unexpected reactions. As a result, machine learning model 102 can generate a recommendation for display on at least one computer network 108 to minimize the occurrence of the unexpected reactions to medication X. For example, the recommendation can state, “A patient being administered both medication Y and medication X can lead to unexpected reactions. Use caution and closely monitor the patient if administering both.”

The recommendation can be displayed to increase an occurrence of the healthcare service or the healthcare condition. For example, dataset 204 can be indicative of a healthcare service of a patient recovering from illness A in five days when administered medication A. Machine learning model 102 can determine that dataset 204 correlates to a first healthcare condition of patients on average recovering from illness A in five days when administered medication A. Machine learning model 102 can compare the first healthcare condition to a second healthcare condition of patients on average recovering from illness A in eight days when administered medication B. As a result, machine learning model 102 can generate a recommendation for display on at least one computer network 108 to increase the occurrence of patients being administered medication A to treat the illness as opposed to medication B. For example, the recommendation can state, “A patient with illness A on average recovers in five days when administered medication A. While a patient with illness A on average recovers in eight days when administered medication B. It is recommended for a faster recovery to prescribe a patient medication A instead of medication B.”

Dataset 204 can be received from a first healthcare computer network associated with a first computer network 108 and the recommendation can be provided for display on first computer network 108. For example, dataset 204 can be indicative of a healthcare service of a clinician of a first healthcare computer network associated with first computer network 108 administering medication A when they intended to administer medication B. Upon comparing dataset 204 to clinical data 202 and previously received datasets 204, machine learning model 102 can determine that dataset 204 correlates to a healthcare condition of other clinicians of the first healthcare computer network associated with first computer network 108 having the same incident. Thus, machine learning model 102 can identify that clinicians administering medication A when they intended to administer medication B is a trend at the first healthcare computer network. As a result, machine learning model 102 can generate a recommendation for display on the at least one computing device 110 of first computer network 108. For example, the recommendation can say, “There is a trend of clinicians administering medication A when they intend to administer medication B. It is recommended to create new labels for medication A to further differentiate the medications.”

Dataset 204 can be received from a first healthcare computer network associated with a first computer network 108 and the recommendation can be provided for display on first computer network 108 or second computer network 108. For example, dataset 204 can be indicative of a healthcare service of a clinician of the first healthcare computer network associated with first computer network 108 administering medication A when they intended to administer medication B. Upon comparing dataset 204 to clinical data 202 and previously received datasets 204, machine learning model 102 can determine that dataset 204 correlates to a healthcare condition of clinicians of a second healthcare computer network associated with second computer network 108 having the same incident. Thus, machine learning model 102 can identify that clinicians administering medication A when they intended to administer medication B is a trend at the first healthcare computer network and the second healthcare computer network. As a result, machine learning model 102 can generate a recommendation for display on at least one computing device 110 of first computer network 108 and at least one computing device 110 of second computer network 108. Machine learning model 102 can generate a first recommendation for display on first computer network 108 and a second recommendation for display on second computer network 108, the first recommendation different than or the same as (e.g., identical to) the second recommendation.

First dataset 204 can be received from a first healthcare computer network associated with a first computer network 108 and second dataset 204 can be received from a second healthcare computer network associated with a second computer network 108. Machine learning model 102 can determine that first dataset 204 or second dataset 204 correlate to a first healthcare condition or a second healthcare condition. As a result, machine learning model 102 can generate a first recommendation or a second recommendation to be displayed on first computer network 108 or second computer network 108.

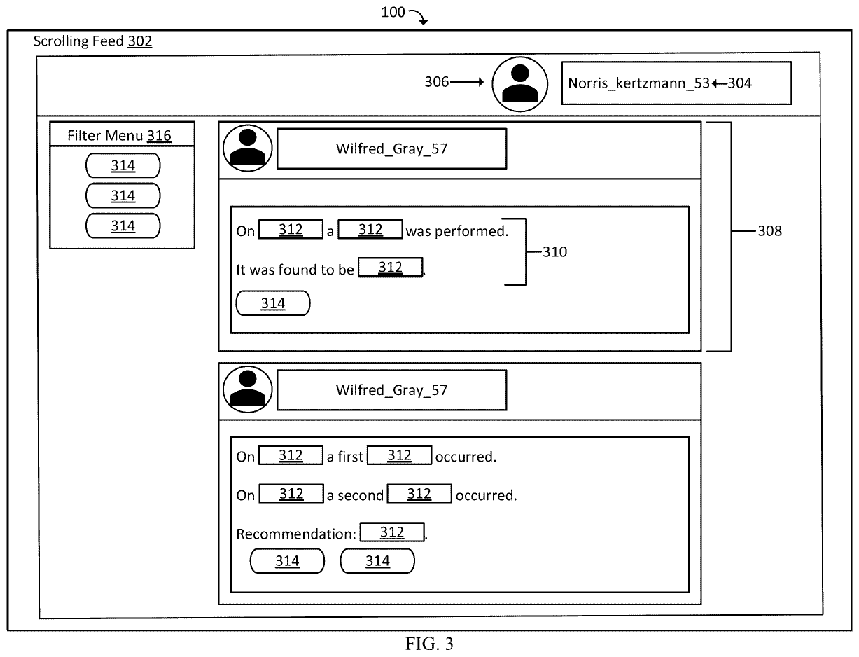

Action 206 can include a post in a scrolling feed. The post and the scrolling feed are described in more detail in the description of FIG. 3, below. System 100 can assign at least one permission set to at least one clinician associated with at least one computer network 108. The permission set assigned to each clinician can be associated with a role of the clinician. For example, an admin of a first healthcare computer network associated with at least one computer network 108 can have greater permissions than a nurse at the first healthcare computer network. The permission set assigned to each clinician can give each clinician different access to data processing system 101 and the various components, inputs, and outputs of system 100. The permission set can include various subset permission sets, each subset permission set with less access than the permission set. For example, an admin can include a first permission set and can have access to all components, inputs, and outputs of system 100 while a nurse with a subset of the first permission set can only have access to the scrolling feed.

System 100 can transmit action 206 to at least one clinician of at least one computer network 108. For example, system 100 can transmit action 206 to a first clinician of first computer network 108, the first clinician including a first permission set. The first permission set giving the first clinician access to action 206. Action 206 can then be reviewed by the first clinician. For example, if action 206 is a recommendation, the first clinician can review the recommendation to approve or deny the recommendation. In response to the first clinician denying the recommendation, machine learning model 102 can generate a new action 206 (e.g., a new recommendation), the first clinician can edit one or more words of the recommendation, or the first clinician can generate their own recommendation. In response to the clinician approving the recommendation, the clinician can transmit a notice of approval for action 206.

System 100 can receive, from at least one clinician, a notice of approval for action 206. For example, in response to a first clinician approving action 206, the first clinician can transmit a notice of approval for action 206 to system 100.

System 100 can transmit action 206 to a second clinician. For example, in response to receiving a notice of approval from a first clinician, action 206 can be transmitted to a second clinician. The second clinician can include a second permission set and the first clinician can include a first permission set. The second permission set can include a subset of the first permission set. For example, in response to a healthcare computer network admin approving a recommendation stating, “There is a trend of clinicians administering medication A when they intend to administer medication B. It is recommended to create new labels for medication A to further differentiate the medications.” The recommendation can be transmitted to a nurse of the healthcare computer network to implement the recommendation.

System 100 can receive feedback from at least one computing device 110 of at least one computer network 108. The feedback can be received from first computing device 110 or second computing device 110. The feedback can be received in response to causing at least one computing device 110 of at least one computer network 108 to perform action 206. For example, the feedback can be received in response to causing first computing device 110 of first computer network 108 and second computing device 110 of second computer network 108 to display a recommendation.

The feedback can take the form of textual or voice feedback. The feedback can include explicit feedback, for example, direct ratings from the clinician (e.g., thumbs up or thumbs down or star ratings). The feedback can include implicit feedback gathered from the clinician's behavior on computing device 110 or the scrolling feed (e.g., click-through rates, time spent on page, interaction patterns, inferences on user preferences, etc.). The feedback can include corrective feedback, such as the clinician correcting errors in action 206. For example, changes made by the clinician reviewing action 206 can be included in the corrective feedback. The feedback can include descriptive feedback such as detailed comments or suggestions from at least one clinician. For example, action 206 can include a recommendation for clinicians to prescribe medication A in place of medication B to treat illness A. The feedback can then indicate if the clinicians saw better treatment of illness A with medication A than they had seen with medication B.

System 100 can retrain machine learning model 102 using the feedback. For example, machine learning model 102 can be retrained using the feedback, clinical data 202, or dataset 204. Multiple iterations of retraining can be performed until a predetermined accuracy or precision is achieved by machine learning model 102 to ensure accurate generation of action 206.

FIG. 3, among others, depicts an example of system 100 of identifying healthcare conditions common to multiple separate healthcare computer networks (e.g., computer network 108). System 100 can include at least one scrolling feed 302. Scrolling feed 302 can be accessible by at least one computing device 110 of at least one computer network 108. Scrolling feed 302 can be accessed by at least one clinician of at least one computer network 108 given their permission set includes access to scrolling feed 302. Each computer network 108 can include a unique scrolling feed 302. For example, first computer network 108 can include a first scrolling feed 302 and second computer network 108 can include second scrolling feed 302.

System 100 can receive at least one access request. The access request can be for access to one or more components, inputs, or outputs of system 100. For example, the access request can be for access to scrolling feed 302. The access request can be received from at least one computing device 110 of at least one computer network 108. The access request can be associated with at least one clinician of computer network 108. For example, the access request can include a request made by a first clinician to access machine learning model 102 or scrolling feed 302. The access request can include an email associated with the clinician, a request to onboard the clinician with machine learning model 102, or a request for at least one identifier 304.

System 100 can receive a first access request from first computing device 110 of first computer network 108 and a second access request from second computing device 110 of second computer network 108. The first access request can be associated with a first clinician of first computer network 108 and the second access request can be associated with a second clinician of second computer network 108. The first access request can include a first email address and a request to onboard the first clinician and the second access request can include a second email address and a request to onboard the second clinician.

System 100 can verify at least one clinician. A clinician can be verified as being employed by at least one healthcare computer network associated with at least one computer network 108. The clinician can be verified using an email address. For example, if the clinician is employed by a healthcare computer network, they can have an employee email address. System 100 can have access to at least one database including email addresses of clinicians employed by the hospitals associated with computer networks 108. For example, in response to receiving an access request including an email address of a clinician, system 100 can access the database to verify that the clinician is an employee of at least one hospital associated with computer network 108.

System 100 can generate at least one identifier 304. Identifier 304 can be generated for at least one clinician. For example, in response to receiving an access request for a clinician, the data processing system 101 (or other computing device 110) can generate and provide identifier 304 to the clinician. In response to receiving an access request for the clinician and verifying the clinician, identifier 304 can be generated for the clinician. Each clinician can be given at least one identifier 304 to anonymize their identity. For example, system 100 can generate a first identifier 304 for a first clinician and a second identifier 304 for a second clinician, first identifier 304 can be different from the second identifier 304. Identifier 304 can include a randomly generated series of numbers, letters, and special characters. Identifier 304 can be randomly selected from a list of pre-generated identifiers 304. For example, the list can include identifiers 304 such as “sodif02n”, “coipa129”, and “Norris_kertzmann_53”. As a result, a clinician can then be randomly assigned the “sodif02n” identifier 304.

System 100 can generate at least one profile picture 306. Profile picture 306 can be generated for each clinician and can be generated along side identifier 304. For example, in response to receiving an access request for a clinician, identifier 304 and profile picture 306 can be generated for the clinician. In response to receiving an access request for the clinician and verifying the clinician, profile picture 306 can be generated for the clinician. Each clinician can be given at least one profile picture 306 to anonymize their identity. For example, system 100 can generate a first profile picture 306 for a first clinician and a second profile picture 306 for a second clinician, first profile picture 306 different from second profile picture 306. Profile picture 306 can include an image generated by machine learning model 102 or another machine learning or artificial intelligence model trained to generate images.

Upon receiving identifier 304, a clinician can request a new identifier 304. This can result in a new randomly generate identifier 304 or a new identifier 304 being selected from the list of pre-generated identifiers 304. Upon receiving profile picture 306, a clinician can request a new profile picture 306. This can result in a new image being generated by a machine learning model (e.g., machine learning model 102) or an artificial intelligence model.

Upon being verified, receiving identifier 304, and receiving profile picture 306, a clinician can access scrolling feed 302, given the clinician's permission set includes access to scrolling feed 302. For example, a first clinician of a first healthcare computer network can access scrolling feed 302 using a first computing device 110 of a first computer network 108. Scrolling feed 302 can be used by the clinician to view at least one post 308.

System 100 can generate at least one post 308. Post 308 can be part of scrolling feed 302. Action 206 can include generating at least one post 308. Post 308 can include a profile picture 306 or an identifier 304 associated with one or more clinicians. Post 308 can be associated with clinical data 202, dataset 204, or a data template generated form dataset 204. Scrolling feed 302 can include a first post 308 and a second post 308. First post 3085 can be associated with a first dataset 204 and second post 308 can be associated with a second dataset 204.

Post 308 can include at least one body 310. Body 310 of post 308 can include a recommendation generated by machine learning model 102. Body 310 can include a summary of clinical data 202, dataset 204 or a data template generated from dataset 204. Body can include one or more data fields 312 extracted from clinical data 202, dataset 204, or the data template. Data fields 312 can include a date, a time, a healthcare service, a healthcare condition, or a status. Data fields 312 can include any values from clinical data 202, dataset 204 or a data template generated from dataset 204. For example, body 310 of a post can state, “On December 5^{th} a new physical therapy was performed. It was found to be successful.” With “December 5^{th}”, “new physical therapy”, and “successful” being examples of data fields 312.

Post 308 can include at least one tag 314. Tag 314 can summarize key information from post 308. Tag 314 can be generated by machine learning model 102. Tag 314 can be generated based on clinical data 202, dataset 204, or a data template associated with post 308. Tag 314 can include at least one term extracted form clinical data 202, dataset 203, or the data template associated with post 308. For example, post 308 can be associated with dataset 204 indicative of a healthcare service of a patient receiving a new form of physical therapy. Machine learning model 102 can analyze dataset 204 and determine tag 314 of “physical therapy” for post 308 associated with dataset 204. Post 308 can include a first tag 314 and a second tag 314, the second tag 314 different from or the same as (e.g., identical to) first tag 314.

Scrolling feed 302 can include at least one filter menu 316. Filter menu 316 can provide a list of all tags 314 associated with posts 308 on scrolling feed 302. Filter menu 316 can be interacted with by at least one clinician with access to scrolling feed 302. A clinician can select or deselect one or more tags 314 on filter menu 316 to filter by that tag type. For example, a clinician selecting the “physical therapy” tag 314 can result in the only posts 308 with “physical therapy” tags 314 being displayed on scrolling feed 302. A clinician can select a first tag 314 and a second tag 314. This can result in only posts 308 with first tag 314 or second tag 314 being displayed on scrolling feed 302.

System 100 can enable two-way communication between users of different or the same computer networks 108. For example, system 100 can enable communication between a first user of a first computing device 110 of a first computer network 108 and a second user of a second computing device 110 of the first computer network 108. System 100 can enable communication between a first user of a first computing device 110 of a first computer network and a second user of a second computing device 110 of a second computer network 108.

In response to generating post 308 associated with a first user of first computing device 110 of first computer network 108 or causing action 206, system 100 may receive a response from second computing device 110 of second computer network 108. System 100 may add the response to a comment section of post 308. System 100 may transmit the response from first computing device 110 of first computer network 108 to second computing device 110 of second computer network 108. In response to transmitting the response, system 100 may receive a second response from second computing device 110 of second computer network 108, which system 100 may then transmit to from first computing device 110 of first computer network 108.

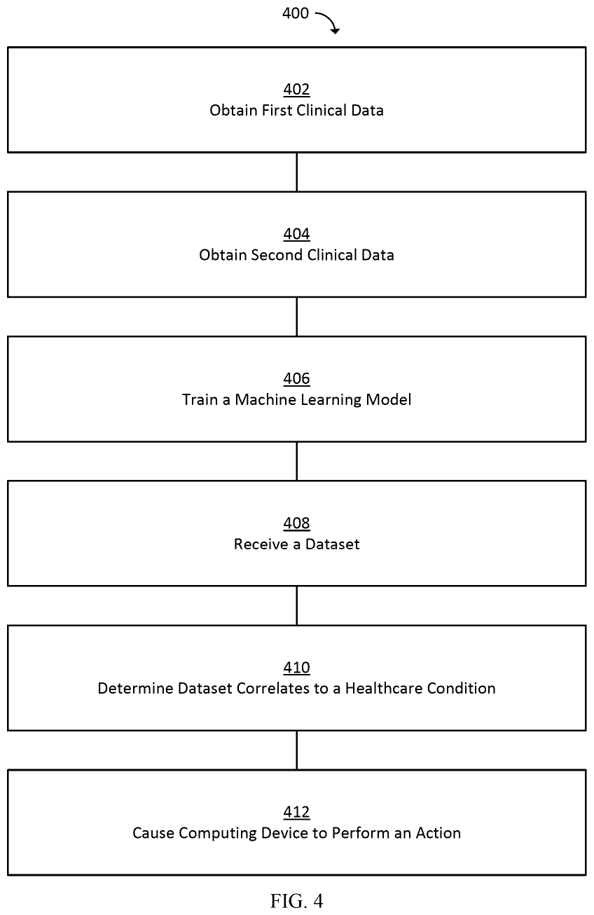

FIG. 4, among others, depicts an example of a method 400 of identifying healthcare conditions common to multiple separate healthcare computer networks. Method 400 can be performed by one or more components of system 100.

Method 400 can include at least one act of obtaining first clinical data 202 (e.g., act 402). First clinical data 202 can be obtained, by one or more processors 106 from at least one computing device 110 of at least one computer network 108. For example, first clinical data 202 can be obtained from at least one computing device 110 of a first computer network 108.

Method 400 can include at least one act of obtaining second clinical data 202 (e.g., act 404). Second clinical data 202 can be obtained, by one or more processors 106 from at least one computing device 110 of at least one computer network 108. For example, second clinical data 202 can be obtained from at least one computing device 110 of a second computer network 108. Second computer network 108 can be separate from first computer network 108.

Method 400 can include at least one act of training at least one machine learning model 102 (e.g., act 406). Machine learning model 102 can be trained by one or more processors 106 using clinical data 202. For example, machine learning model 102 can be trained by one or more processors 106 using first clinical data 202 and second clinical data 202 to identify a plurality of healthcare conditions.

Method 400 can include at least one act of receiving at least one dataset 204. Dataset 204 can be received from at least one computer network 108. For example, dataset 204 indicative of at least one healthcare service can be received by one or more processors 106 from first computer network 108 or second computer network 108.

Method 400 can include receiving by one or more processors 106 from first computer network 108 first dataset 204 indicative of a first healthcare service. Method 400 can include receiving by one or more processors 106 from second computer network 108 second dataset 204 indicative of a second healthcare service. The first healthcare service can be different from the second healthcare service. The first healthcare service can be the same as (e.g., identical to) the second healthcare service.

Method 400 can include receiving by one or more processors 106 from third computer network 108 second dataset 204. Third computer network 108 can be separate from first computer network 108 and second computer network 108.

Method 400 can include at least one act of determining that at least one dataset 204 correlates to a first healthcare condition (e.g., act 410). For example, one or more processors 106 can determine using machine learning model 102 that dataset 204 indicative of the healthcare service correlates to a first healthcare condition of the plurality of healthcare conditions.

Method 400 can include determining first dataset 204 correlates to a first healthcare condition and second dataset 204 correlates to the first healthcare condition. For example, one or more processors 106 can determine using machine learning model 102 that first dataset 204 indicative of the first healthcare service and second dataset 204 indicative of the second healthcare service correlates to the first healthcare condition of the plurality of healthcare conditions.

Method 400 can include determining first dataset 204 correlates to a first healthcare condition and second dataset 204 correlates to a second healthcare condition. For example, one or more processors 106 can determine using machine learning model 102 that first dataset 204 indicative of the healthcare service correlates to the first healthcare condition of the plurality of healthcare conditions and that second dataset 204 indicative of the healthcare service correlates to the second healthcare condition of the plurality of healthcare conditions. The first healthcare condition different from or the same as (e.g., identical to) the second healthcare condition.

Method 400 can include at least one act of causing at least one computing device 110 to perform action 206 (e.g., act 412). Responsive to determining that dataset 204 indicative of at least one healthcare service correlates to at least one healthcare condition, method 400 can cause at least one computing device 110 of at least one computer network 108 to perform action 206. For example, one or more processors 106 can, responsive to determining that dataset 204 indicative of the healthcare service correlates to the first healthcare condition, cause first computing device 110 of first computing network 108 and second computing device 110 of second computing network 108 to perform an action.

Method 400 can include causing first computing device 110 to perform a first action 206. For example, one or more processors 106 can, responsive to determine that first dataset 204 indicative of the healthcare service correlates to the first healthcare condition, cause first computing device 110 of first computer network 108 to perform first action 206.

Method 400 can include causing second computing device 110 to perform a second action 206. For example, one or more processors 106 can, responsive to determine that second dataset 204 indicative of the healthcare service correlates to the second healthcare condition, cause second computing device 110 of second computer network 108 to perform second action 206. Second action 206 can be the same as (e.g., identical to) or different from the first action 206.

Method 400 can include causing third computing device 110 to perform at least one action 206. For example, one or more processors 106 can, responsive to determine that second dataset 204 indicative of the healthcare service correlates to the first healthcare condition or second healthcare condition, cause third computing device 110 of third computer network 108 to perform at least one action 206.

Method 400 can include at least one computing device 110 of at least one computer network 108 executing at least one action 206. For example, first action 206 can be executed by first computing device 110 of first computer network 108 and second action 206 can be executed by second computing device 110 of second computer network 108. First action 206 can be identical to or different than second action 206.

Method 400 can receive feedback from at least one computing device 110. One or more processors 106 can, responsive to causing at least one computing device 110 of at least one computing network 108 to perform action 206, receive feedback from at least one computing device 110. For example, one or more processors 106 can, responsive to causing first computing device 110 of first computer network 108 and second computing device 110 of second computer network 108 to perform action 206, receive feedback from first computing device 110 or second computing device 110. Machine learning model 102 can be retrained by one or more processors 106 using the feedback.

FIG. 5, among others, depicts an example of system 100 of identifying healthcare conditions common to multiple separate healthcare computer networks (e.g., computer network 108). System 100 can include at least one computing system 500. Computing system 500 can include or be used to implement system 100, or its components such as computing device 110. Computing system 500 can be or include the data processing system 101. Computing system 500 can include at least one data bus 505 or other communication component for communicating information and a processor 106 or processing circuit coupled with data bus 505 for processing information.

Computing system 500 can include at least one processor 106 or processing circuits coupled with data bus 505 for processing information. Computing system can include at least one memory 104, such as random-access memory (RAM) or other dynamic storage device, coupled with data bus 505 for storing information and instructions to be executed by processors 106. Memory 104 can also be used for storing position information, temporary variables, or other intermediate information during execution of instructions by processor 106. Computing system 500 can include read only memory (ROM) 515 or other static storage devices coupled with data bus 505 for storing static information and instructions for processor 106. Computing system 500 can include at least one storage device 520. Storage device 520 can include a solid-state device, magnetic disk, or optical disk and can be coupled with data bus 505 to persistently store information and instructions. Storage device 520 can include at least one data repository.

Computing system 500 can include at least one input device 530. Input device 503 can include a touch screen display or a cursor control (e.g., a mouse, a trackball, cursor direction keys) for communicating direction information and command selections to processor 106 as well as controlling cursor movement of the display. The display can be part of computing device 110 or another component of FIG. 1, for example.

The processes and methods described herein can be implemented by computing system 500 in response to processor 106 executing an arrangement of instructions contained in memory 104. Such instructions can be read into memory 104 form another computer-readable medium, such as storage device 520. Execution of the arrangement of instructions contained in memory 104 can cause computing system 500 to perform the illustrative process described herein. One or more processors 106 in a multi-processing arrange can also be employed to execute the instructions contained in memory 104. Hard-wired circuitry can be used in place of or in combination with software instructions together with the systems and methods described herein. Systems and methods described herein are not limited to any specific combination of hardware circuitry and software.

Although an example of computing system 500 has been described in FIG. 5, the subject matter including the operations described in this specification can be implemented in other types of digital electronic circuitry, or in computer software, firmware, or hardware, including the structures disclosed in this specification and their structural equivalents, or in combinations of one or more of them.

While acts or operations can be depicted in the drawings or described in a particular order, such operations are not required to be performed in the particular order shown or described, or in sequential order, and all depicted or described operations are not required to be performed. Actions described herein can be performed in different orders.

Having now described some illustrative implementations, it is apparent that the foregoing is illustrative and not limiting, having been presented by way of example. Features that are described herein in the context of separate implementations can also be implemented in combination in a single embodiment or implementation. Features that are described in the context of a single implementation can also be implemented in multiple implementations separately or in various sub-combinations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular can include implementations including a plurality of these elements, and any references in plural to any implementation or element or act herein can include implementations including only a single element. References in the singular or plural form are not intended to limit the presently disclosed systems or methods, their components, acts, or elements to single or plural configurations. References to any act or element being based on any information, act or element can include implementations where the act or element is based at least in part on any information, act, or element.

References to “or” can be construed as inclusive so that any terms described using “or” can indicate any of a single, more than one, and all of the described terms. References to at least one of a conjunctive list of terms can be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

The systems and methods described herein can be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

Systems and methods described herein can be embodied in other specific forms without departing from the characteristics thereof. For example, descriptions of positive and negative electrical characteristics can be reversed. For example, elements described as negative elements can instead be configured as positive elements and elements described as positive elements can instead by configured as negative elements. Further relative parallel, perpendicular, vertical or other positioning or orientation descriptions include variations within +/−10% or +/−10 degrees of pure vertical, parallel or perpendicular positioning. References to “approximately,” “about” “substantially” or other terms of degree include variations of +/−10% from the given measurement, unit, or range unless explicitly indicated otherwise. Coupled elements can be electrically, mechanically, or physically coupled with one another directly or with intervening elements. Scope of the systems and methods described herein is thus indicated by the appended claims, rather than the foregoing description, and changes that come within the meaning and range of equivalency of the claims are embraced therein.

What is claimed is:

1. A system of identifying healthcare conditions common to multiple separate healthcare computer networks, comprising:
one or more processors coupled with memory to:
obtain, from at least one computing device of a first computer network, first clinical data;
obtain, from at least one computing device of a second computer network, second clinical data, the second computer network separate from the first computer network;
train, using the first clinical data and the second clinical data, a machine learning model to identify a plurality of healthcare conditions;
receive, from the first computer network or the second computer network, a dataset indicative of a healthcare service;
determine, using the machine learning model, that the dataset indicative of the healthcare service correlates to a first healthcare condition of the plurality of healthcare conditions; and
cause, responsive to determining that the dataset indicative of the healthcare service correlates to the first healthcare condition, a first computing device of the first computer network and a second computing device of the second computer network to perform an action.

2. The system of claim 1, comprising the one or more processors coupled with memory to:
convert, responsive to obtaining the first clinical data, the first clinical data to a data format; and
convert, responsive to obtaining the second clinical data, the second clinical data to the data format.

3. The system of claim 1, comprising:
the one or more processors coupled with memory to cause the first computing device of the first computer network and the second computing device of the second computer network to perform the action, wherein the action includes a recommendation provided for display from one of the first computer network or the second computer network to minimize an occurrence of the healthcare service.

4. The system of claim 1, comprising the one or more processors coupled with memory to:
receive, from a third computing device of the first computer network, a first access request associated with a first clinician;
receive, from a fourth computing device of the second computer network, a second access request associated with a second clinician;
generate a first identifier for the first clinician; and
generate a second identifier for the second clinician, the second identifier different from the first identifier.

5. The system of claim 1, comprising the one or more processors coupled with memory to:
transmit the action to a first clinician of the first computer network, the first clinician with a first permission set;
receive, from the first clinician, a notice of approval for the action; and
transmit, responsive to receiving the notice of approval, the action to a second clinician of the second computer network, the second clinician with a second permission set, the second permission set a subset of the first permission set.

6. The system of claim 1, comprising the one or more processors coupled with memory to:
receive, responsive to causing the first computing device of the first computer network and the second computing device of the second computer network to perform the action, feedback from the first computing device or the second computing device; and
retrain, using the feedback, the machine learning model.

7. The system of claim 1, wherein the action is a first action, comprising:
the first computing device of the first computer network to perform the first action; and
the second computing device of the second computer network to perform a second action, the first action different than the second action.

8. The system of claim 1, wherein the action is a first action, comprising:
the first computing device of the first computer network to perform the first action; and
the second computing device of the second computer network to perform a second action, the first action identical to the second action.

9. The system of claim 1, wherein the dataset indicative of the healthcare service is a first dataset indicative of a first healthcare service, comprising the one or more processors coupled with memory to:
receive, from the first computer network, the first dataset indicative of the first healthcare service;
receive, from the second computer network, a second dataset indicative of a second healthcare service; and
determine, using the machine learning model, that the first dataset indicative of the first healthcare service and the second dataset indicative of the second healthcare service correlate to the first healthcare condition of the plurality of healthcare conditions.

10. The system of claim 1, comprising the one or more processors coupled with memory to:
identify, responsive to receiving the dataset indicative of the healthcare service, a data template based on the dataset indicative of the healthcare service;
extract, from the dataset indicative of the healthcare service, key terms;
populate the data template with the key terms;
determine, using the machine learning model, that the data template correlates to the first healthcare condition of the plurality of healthcare conditions; and
cause, responsive to determining that the data template correlates to the first healthcare condition, the first computing device of the first computer network and the second computing device of the second computer network to perform the action.

11. The system of claim 1, wherein the dataset indicative of the healthcare service is a first dataset indicative of a first healthcare service, comprising the one or more processors coupled with memory to:
receive, from a third computer network separate from the first computer network and the second computer network, a second dataset indicative of a second healthcare service;
determine, using the machine learning model, that the second dataset indicative of the second healthcare service correlates to the first healthcare condition of the plurality of healthcare conditions; and
cause, responsive to determining that the second dataset indicative of the healthcare service correlates to the first healthcare condition, a third computing device of the third computer network to perform the action.

12. The system of claim 1, comprising the one or more processors coupled with memory to:
identify, responsive to receiving the dataset indicative of the healthcare service, information missing from the dataset indicative of the healthcare service;
transmit, to the first computer network or the second computer network, a request for the information missing from the dataset indicative of the healthcare service;
receive, from the first computer network or the second computer network, the information missing from the dataset indicative of the healthcare service; and
populate the dataset indicative of the healthcare service with the information missing from the dataset indicative of the healthcare service.

13. The system of claim 1, wherein the dataset indicative of the healthcare service is a first dataset indicative of a first healthcare service and the action is a first action, comprising the one or more processors coupled with memory to:
receive, from the first computer network, the first dataset indicative of the first healthcare service;
receive, from the second computer network, a second dataset indicative of a second healthcare service;
determine, using the machine learning model, that the first dataset indicative of the healthcare service correlates to the first healthcare condition of the plurality of healthcare conditions and the second dataset indicative of the healthcare service correlates to a second healthcare condition of the plurality of healthcare conditions, the first healthcare condition different from the second healthcare condition;
cause, responsive to determining that the first dataset indicative of the healthcare service correlates to the first healthcare condition, the first computing device of the first computer network to perform the first action; and
cause, responsive to determining that the second dataset indicative of the healthcare service correlates to the second healthcare condition, the second computing device of the second computer network to perform a second action.

14. A method of identifying healthcare conditions common to multiple separate healthcare computer networks, comprising:
obtaining, by one or more processors from at least one computing device of a first computer network, first clinical data;
obtaining, by the one or more processors from at least one computing device of a second computer network, second clinical data, the second computer network separate from the first computer network;
training, by the one or more processors using the first clinical data and the second clinical data, a machine learning model to identify a plurality of healthcare conditions;
receiving, by the one or more processors from the first computer network or the second computer network, a dataset indicative of a healthcare service;
determining, by the one or more processors using the machine learning model, that the dataset indicative of the healthcare service correlates to a first healthcare condition of the plurality of healthcare conditions; and
causing, by the one or more processors responsive to determining that the dataset indicative of the healthcare service correlates to the first healthcare condition, a first computing device of the first computer network and a second computing device of the second computer network to perform an action.

15. The method of claim 14, wherein the action is a first action, comprising:
executing, by the first computing device of the first computer network, the first action; and
executing, by the second computing device of the second computer network, a second action, the first action different than the second action.

16. The method of claim 14, wherein the action is a first action, comprising:
executing, by the first computing device of the first computer network, first action; and
executing, by the second computing device of the second computer network to perform a second action, the first action identical to the second action.

17. The method of claim 14, wherein the dataset indicative of the healthcare service is a first dataset indicative of a first healthcare service, comprising:
receiving, by the one or more processors from the first computer network, the first dataset indicative of the first healthcare service;
receiving, by the one or more processors from the second computer network, a second dataset indicative of a second healthcare service; and
determining, by the one or more processors using the machine learning model, that the first dataset indicative of the first healthcare service and the second dataset indicative of the second healthcare service correlate to the first healthcare condition of the plurality of healthcare conditions.

18. The method of claim 14, comprising:
receiving, by the one or more processors responsive to causing the first computing device of the first computer network and the second computing device of the second computer network to perform the action, feedback from the first computing device or the second computing device; and
retraining, by the one or more processors using the feedback, the machine learning model.

19. The method of claim 14, wherein the dataset indicative of the healthcare service is a first dataset indicative of a first healthcare service, comprising:
receiving, by the one or more processors from a third computer network separate from the first computer network and the second computer network, a second dataset indicative of a second healthcare service;
determining, by the one or more processors using the machine learning model, that the second dataset indicative of the second healthcare service correlates to the first healthcare condition of the plurality of healthcare conditions; and
causing, by the one or more processors responsive to determining that the second dataset indicative of the healthcare service correlates to the first healthcare condition, a third computing device of the third computer network to perform the action.

20. The method of claim 14, wherein the dataset indicative of the healthcare service is a first dataset indicative of a first healthcare service and the action is a first action, comprising:
receiving, by the one or more processors from the first computer network, the first dataset indicative of the first healthcare service;
receiving, by the one or more processors from the second computer network, a second dataset indicative of a second healthcare service;
determining, by the one or more processors using the machine learning model, that the first dataset indicative of the healthcare service correlates to the first healthcare condition of the plurality of healthcare conditions and the second dataset indicative of the healthcare service correlates to a second healthcare condition of the plurality of healthcare conditions, the first healthcare condition different from the second healthcare condition;
causing, by the one or more processors responsive to determining that the first dataset indicative of the healthcare service correlates to the first healthcare condition, the first computing device of the first computer network to perform the first action; and
causing, by the one or more processors responsive to determining that the second dataset indicative of the healthcare service correlates to the second healthcare condition, the second computing device of the second computer network to perform a second action.
